(19) United States
(12) Patent Application Publication
(10) Pub. No.: US 2024/0180210 A1
(43) Pub. Date: Jun. 6, 2024

(54) PRODUCTION LINE ASSEMBLY AND PRODUCTION LINE FOR LASER WALL BREAKING OF PLANT SPORES

(71) Applicant: HUNAN YUANKE ENGINEERING TECHNOLOGY CO., LTD., Changsha, Hunan (CN)

(72) Inventors: Dongbo LIU, Changsha, Hunan (CN); Jinliang MA, Changsha, Hunan (CN); Yueying WANG, Changsha, Hunan (CN); Huifen ZHAO, Changsha, Hunan (CN); Gaoping HU, Changsha, Hunan (CN); Chang LIU, Changsha, Hunan (CN); Juanjuan CAI, Changsha, Hunan (CN)

(73) Assignees: HUNAN YUANKE ENGINEERING TECHNOLOGY CO., LTD., Changsha, Hunan (CN); SANMENXIA CHANGZHOU BIOTECHNOLOGY CO., LTD., Sanmenxia City, Henan (CN)

(21) Appl. No.: 18/552,315

(22) PCT Filed: Feb. 16, 2022
(86) PCT No.: PCT/CN2022/076428

§ 371 Date: Sep. 25, 2023

(30) Foreign Application Priority Data
Sep. 3, 2021 (CN) 202111031596.8
Dec. 6, 2021 (CN) 202123105962.8

(51) Int. Cl. A23L 5/20; A23L 3/28; A23L 5/30

(52) U.S. Cl. CPC A23L 5/21; A23L 3/28; A23L 5/36

(57) ABSTRACT

A production line assembly for laser wall breaking of plant spores includes a material conveying module, a laser wall breaking module, and a stock solution recycling module which are successively connected by means of pipelines. The material conveying module is used for conveying a suspension liquid to be subjected to laser wall breaking to the laser wall breaking module; the laser wall breaking module is formed by a plurality of light energy receiving regions being successively connected along the conveying direction of the suspension liquid; each light energy receiving region comprises a laser, a light energy receiver, and an XY limiter; the suspension is circulated in the light energy receivers, and the lasers are in one-to-one correspondence with the light energy receivers and are used for performing laser wall breaking on the suspension liquid; the lasers are used for irradiating the light energy receivers horizontally or from top to bottom; and the light energy receivers are mounted on the XY limiters, and the XY limiters are used for adjusting the positions of the light energy receivers in X- axis and Y- axis directions.

### TECHNICAL FIELD

The invention relates to the technical field of laser wall breaking, in particular to a production line assembly and production line for laser wall breaking of plant spores.

### BACKGROUND

The inclusion of spores is not only a source of life for plants, but also a micro-nutrient treasure house. The inclusion of the spores is in a free state, and the size of the free matters is nano-scale tiny particles, and they are easy to absorb by a human body. In order to obtain sufficient access to the contents of the spores, it is constantly sought to break the hard exine shells of the spores.

In the existing wall breaking method, a very important problem of how to reduce the nutrient loss of the content of the spore in the wall breaking process is ignored.

With the rapid development of laser application technology, the technology of breaking the walls of the medicinal plant spores and pollen spores by using laser is also developed, but the problem of how to improve the wall breaking efficiency as much as possible and how to prevent the nutrients in the spores from being damaged to the maximum extent by using the laser wall breaking method is the problem of widely popularizing and using the laser for breaking the walls of the medicinal plant spores and pollen spores. However, when laser is applied to the technology of breaking the walls of spores, such as the technology of non-oxidative breaking the walls of ganoderma spores, it is necessary to consider the destruction of active substances in spore inclusions, and the destruction is reflected in the following two aspects:
(1) the thermal effect of the laser over-irradiation causes burning, charring, vaporization of the spore inclusions.
(2) The light effect of the laser irradiation for a long time causes destructive effects such as stimulation, inhibition and decomposition on spore inclusions.

In view of this, the present invention provides a production line assembly for laser wall breaking of plant spores and a production line for use in a light energy receiving area in a non-oxidation laser wall breaking production line of plant spores.

### DISCLOSURE OF INVENTION

In order to solve the above problems in the prior art, the present invention aims to provide a production line assembly and a production line for laser wall breaking of plant spores.

The technical scheme adopted by the invention is as follows:
a production line assembly for laser wall breaking of plant spores comprises a material conveying module, a laser wall breaking module and a stock solution recovery module which are successively connected through pipelines; the material conveying module is used for conveying the suspension to be subjected to laser wall breaking to the laser wall breaking module;
the laser wall breaking module is formed by sequentially connecting a plurality of light energy receiving areas along the conveying direction of the suspension to be subjected to laser wall breaking;
the light energy receiving area comprises a laser, a light energy receiver and an XY limiter; the suspension liquid to be subjected to laser wall breaking flows in the light energy receiver, and the laser correspond to the light energy receiver one by one and is used for performing laser wall breaking on the suspension liquid to be subjected to laser wall breaking; the laser is used for horizontally irradiating the light energy receiver, or the laser is used for irradiating the light energy receiver from top to bottom;
the light energy receiver is mounted on the XY limiter, and the XY limiter is used for adjusting the position of the light energy receiver in X-axis and Y-axis direction.

Preferably, the material conveying module comprises a material preparation barrel and a conveying pipe, and the conveying pipe is used for extracting the suspension to be subjected to laser wall breaking in the material preparation barrel and conveying the suspension to the light energy receiver; the light energy receiver comprises a turbulence generator, a light energy receiving glass tube and an isolation box type foam isolator which are sequentially connected along the conveying direction of the suspension to be subjected to laser wall breaking.

Preferably, the turbulence generator comprises turbulence promoter and peristaltic pump, the turbulence promoter, the light energy receiving glass tube and the isolation box type foam isolator are sequentially communicated and integrally arranged, one side of the peristaltic pump is connected with turbulence promoter through a pipeline, the other side of peristaltic pump is connected with conveying pipe, which is used for conveying the suspension to be subjected to laser wall breaking into the turbulence promoter.

Preferably, the turbulence promoter comprises a box body, a turbulence promoting chamber and a first water tank are arranged in the box body, a through hole is arranged between the first water tank and the turbulence promoting chamber, the first water tank is connected with one end of the light energy receiving glass tube and is communicated with the turbulence promoting chamber through the through hole, the turbulence promoting chamber is provided with a feed port relative to the other end of the through hole, and the pump is connected with the feed port.

Preferably, partition plates are arranged in the turbulence promoting chamber, and the partition plates comprise the first partition plate and second partition plate which are respectively fixed on two opposite sides of the inner wall of the turbulence promoting chamber, and the first partition plate and the second partition plate are respectively provided in plurality and are alternately arranged between the feed port of the turbulence promoting chamber and the perforation.

Preferably, the turbulence promoting chamber is provided with raised stripes inside, the crisscross setting of raised stripes is in the opposite sides of the inner wall of turbulence promotion chamber, the cross section of the raised stripes is triangle-shaped and arranged between feed port and the perforation of turbulence promotion chamber.

Preferably, the isolation box type foam isolator comprises a second water tank and a foam isolation chamber, a through hole is formed between the second water tank and the foam isolation chamber, one end of the light energy receiving glass tube is connected with the first water tank, the other end of the light energy receiving glass tube is connected with the second water tank, the second water tank is communicated with the foam isolation chamber through the through hole, and a foam outlet and a finished product stock solution outlet are arranged in the foam isolation chamber.

The invention also provides a production line for laser wall breaking of the plant spores, which comprises a raw material inspection and disinfection module, a material preparation module, a production line assembly and an inspection and subpackage module.

Preferably, the raw materials inspection and disinfection module is used for inspecting, disinfecting and storing plant spore raw materials, the material preparation module is used for mixing plant spore and pure water, and the suspension to be subjected to laser wall breaking is prepared, and the inspection and subpackage module is used for inspecting, subpackaging to the finished product stock solution after the broken wall.

Preferably, the raw material inspection and disinfection module comprises a raw material inspection room, a disinfection room and a storage warehouse, and the plant spore raw material is inspected by the raw material inspection room, conveyed to the disinfection room for sterilization and disinfection, and finally conveyed to the storage warehouse for storage;
the inspection and subpackage module comprises a finished product inspection room, a subpackaging room and a finished product storehouse, and after the finished product stock solution subjected to wall breaking is inspected to be qualified by the finished product inspection room, the finished product stock solution is respectively subpackaged in the subpackaging room and finally conveyed to the finished product storehouse for storage.

The beneficial effects of the invention are as follows:
1. modular components are adopted, each module is linearly arranged to build a basic structure production line, and then a production line assembly of the building block type superposed base structure production line is adopted to enlarge the capacity; the basic structure production line completely determines the annual production capacity of wall-broken plant spores by users, and flexible resource allocation is realized.
2. Except that the laser wall breaking module has the possibility of replacing a laser to deal with different types of spores, the rest five modules are completely universal and are equipment, instruments and devices with longer working life.
3. The production line assembly is convenient to carry out small-batch production, the production capacity is increased through the building block type superposed base structure production line assembly, and the original base structure production line is not required to be reformed.
4. Because the spore stock solution after wall breaking flows in a closed environment until subpackaging and is not exposed in the air at all, there is no special requirements for factory buildings and working environments.
5. The factory built according to the scheme is a green factory without dust; no discharge; no noise exists; the energy consumption is extremely low.
6. The construction method of rolling investment without repeated construction brings the difficult problems of overcoming insufficient capital stock and wanting to develop for small micro-enterprises and brings vitality for the future of the small micro-enterprises.
7. The invention is suitable for small and micro enterprises planting plant spore production areas to realize a whole industrial chain forming links of large-scale planting, processing, warehouse logistics, new product research and development, sale and the like. It is beneficial to developing local functional agriculture and developing functional products and health products.

### BRIEF DESCRIPTION OF THE DRAWINGS

The invention is described in further detail below with reference to the accompanying drawings and specific embodiments.

FIG. 1 is a schematic structural diagram of example 1, which is mainly used for showing the structure of a light energy receiving area when a laser is horizontally irradiated;

FIG. 2 is a schematic structure diagram of the example 1, which is mainly used for embodying the structure of the light energy receiver;

FIG. 3 is a sectional view of example 1, mainly used for embodying the structure of the turbulence generator;

FIG. 4 is a schematic sectional view of example 1, which is mainly used to embody the structure of an insulating box-type foam insulator;

FIG. 5 is a schematic sectional view of example 2, which is mainly used for embodying the structure of the turbulence generator;

FIG. 6 is a schematic sectional view of example 2, which is mainly used for embodying the structure of the turbulence generator;

FIG. 7 is a schematic structural diagram of example 3, which is mainly used for showing the structure of a light energy receiving area when the laser is irradiated from the top;

FIG. 8 is an electron microscope image of pine pollen spore before wall breaking in example 1;

FIG. 9 is an electron micrograph image of pine pollen spores after wall breaking in example 1;

FIG. 10 is an electron micrograph image of rape pollen spores after wall breaking in example 1;

FIG. 11 is an electron micrograph image of rose pollen spores after wall breaking in example 1;

FIG. 12 is a block flow diagram of example 4, which is mainly used for embodying the process flow of the production line;

FIG. 13 is a block diagram of example 4, which is mainly used for embodying the structure of the production line assembly.

In the figure: 1—laser strike line, 2—light energy receiving glass tube, 3—isolation box type foam isolator, 31—foam isolation chamber, 32—second water tank, 4—finished product stock solution outlet, 5—foam outlet, 61—pump, 62—turbulence promoter, 621—first water tank, 622—turbulence promoting chamber, 71—first partition plate, 72—second partition plate, 8—feed port, 9—perforation, 10—through hole, 111—tank body, 112—magnetic stirring device, 113—magnetic stirrer, 12—material input tube, 13—material output tube, 14—laser, 15—foam sediment pipeline, 16—XY limiters, 17—Z limiters, 20—raw material inspection and disinfection module, 21—material preparation module, 22—production line assembly, 221—material conveying module, 222—laser wall breaking module, 2221—laser, 2222—quartz glass tank tube, 2223—turbulence generator, 2224—peristaltic pump, 223—stock solution recovery module 23—inspection and subpackage module.

### DETAILED DESCRIPTION

The invention is further described with reference to the following figures and specific examples.

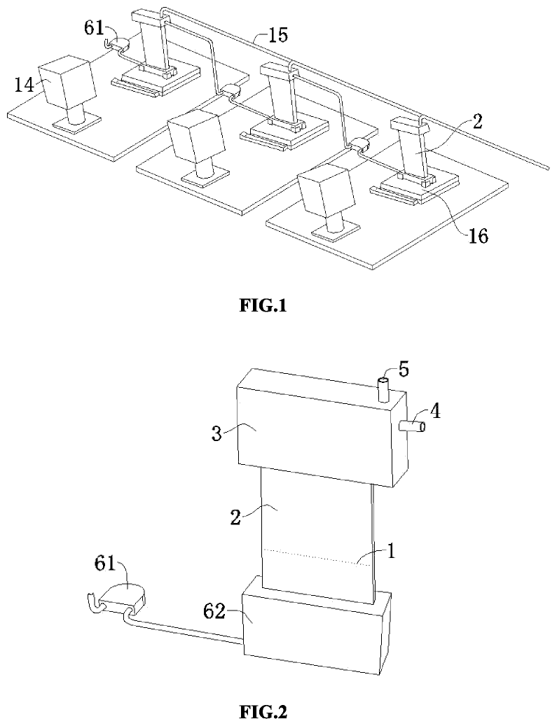

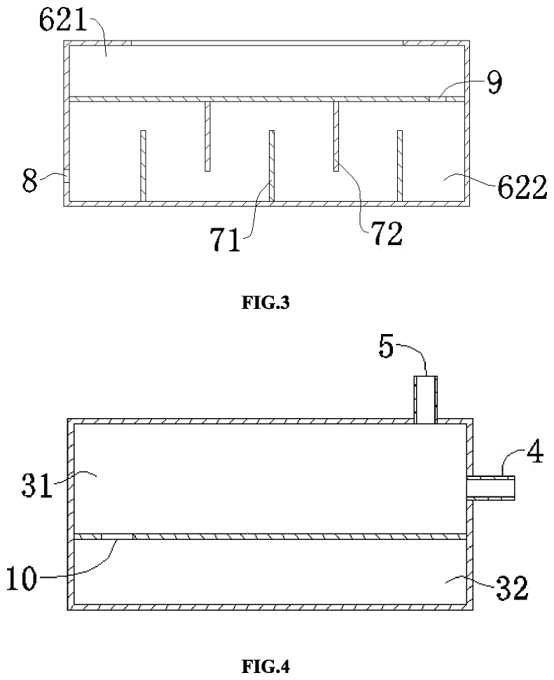

Example 1: a production line assembly for laser wall breaking of plant spores is provided, as shown in FIG. 1-4, comprising a material conveying module 221, a laser wall breaking module 222 and a stock solution recovery module 223 which are successively connected by pipelines; the material conveying module 221 is used for conveying the suspension to be subjected to laser wall breaking to the laser wall breaking module 222;
the laser wall breaking module 222 is formed by sequentially connecting a plurality of light energy receiving areas along the conveying direction of the suspension to be subjected to laser wall breaking; it can be formed by connection, can also be formed by parallel connection, or can be formed by series-parallel connection, as long as a wall breaking production line can be formed.

The light energy receiving area comprises a laser 14, a light energy receiver and an XY limiter 16; the suspension liquid to be subjected to laser wall breaking flows in the light energy receiver, and the laser 14 correspond to the light energy receiver one by one and is used for performing laser wall breaking on the suspension liquid to be subjected to laser wall breaking; the laser 14 is used for horizontally irradiating the light energy receiver, or the laser 14 is used for irradiating the light energy receiver from top to bottom;
the light energy receiver is mounted on the XY limiter 16, and the XY limiter 16 is used for adjusting the position of the light energy receiver in X-axis and Y-axis direction.

In the above technical scheme, the laser wall breaking method is to focus laser beams into laser focuses (light spots) by using lens focusing, and to burn and perforate the spore wall shells by means of the energy of the light spots to break the walls. Therefore, after knowing the laser peak power energy data required for breaking the walls of various spores, the central positioning of the laser spot and the full utilization of the effective light energy are the key points for carrying out nondestructive and efficient wall breaking on the spores. According to the rayleigh effect, generally, the interval of 10% attenuation of the laser 14 from the peak power of a light spot is within 1-2 mm, namely the interval of ±0.5-1.0 mm with the light spot as the center, and spores can receive the most appropriate laser wall breaking energy only when entering the interval. How to realize the accurate positioning of laser spots in a thin and flat light energy receiving glass tube and ensure that the proper laser energy can carry out wall breaking on spores in a nondestructive and efficient manner is an important link in the production line process of a laser wall breaking method. For this purpose, the laser 14, the light energy receiver and the XY limiter 16 are arranged, so that under the guidance of the light guided by the laser 14, the spot center of the laser is accurately positioned at the specified position in the light energy receiver by the XY limiter 16 with the precision displacement of micron order. Laser 14 is fixed in position on the station base table, which is related to the focusing mirror parameters of the laser 14. The displacement stroke of the light energy receiver in the XY direction is controlled by the XY position limiter 16 to match the center positioning of the light spot, wherein the X direction positioning the central vertical line of light energy receiving glass tube 2; the Y direction positioning the light spot at a specified position in the irradiation surface of the thin and flat type light energy receiving glass tube 2.

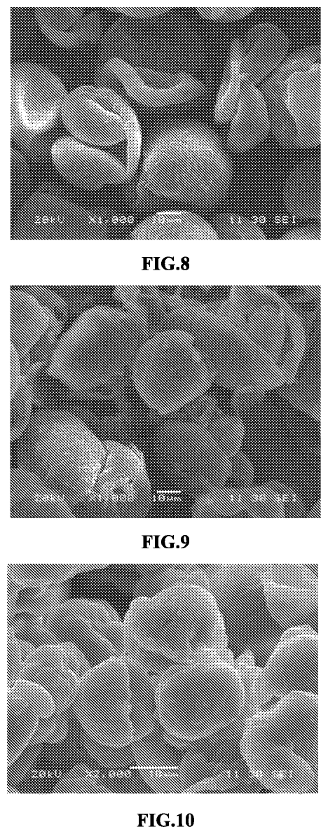

The laser light energy receiving area is applied to pine pollen spore wall breaking, rape pollen spore wall breaking and rose pollen spore wall breaking tests to obtain the final product shown in FIG. 8-11. After wall breaking, pine pollen spores, rape pollen spores and rose pollen spore stock solutions are tested, and the content mg/g of main index substances of spore inclusions is far higher than that of main brand products in the current market.

Referring to FIG. 1-7, the turbulence generator comprises a turbulence promoter 62 and a peristaltic pump 61, the turbulence promoter 62, the light energy receiving glass tube 2 and the isolation box type foam isolator 3 are sequentially communicated and integrally arranged, one side of the peristaltic pump 61 is connected with the turbulence promoter 62 through a pipeline, and the other side of the peristaltic pump 61 is connected with the conveying pipe, which is used for conveying the suspension to be subjected to laser wall breaking into the turbulence promoter 62.

Referring to FIG. 1 to 7, the turbulence promoter 62 comprises a box body, a turbulence promoting chamber 622 and a first water tank 621 are arranged in the box body, a through hole 10 is arranged between the first water tank 621 and the turbulence promoting chamber 622, the first water tank 621 is connected with one end of the light energy receiving glass tube 2 and is communicated with the turbulence promoting chamber 622 through the through hole 10, and the turbulence promoting chamber 622 is provided with a feed port 8 relative to the other end of the through hole 10; the pump 61 is connected with the feed port 8, so that the suspension enters the turbulence promoting chamber 622 and then enters the first water tank 621. Partition plates are arranged in the turbulence promoting chamber 622, and partition plates comprise the first partition plate 71 and the second partition plate72 which are respectively fixed on two opposite sides of the inner wall of the turbulence promoting chamber 622, the first partition 71 and the second partition 72 being fixed on two opposite sides of the inner wall of the turbulence promoting chamber 622, respectively. The first partition plate 71 and the second partition plate 72 are provided in plurality and are alternately arranged between the feed port 8 of the turbulence promoting chamber 622 and the perforations 9.

In the above solution, the turbulence generator is an assembly that controls the fluid velocity, flow rate and fluid form, and consists of a speed adjustable peristaltic pump and a turbulence promoter 62 that is capable of generating turbulence. The low-voltage direct-current speed-regulating peristaltic pump can monitor the flow and the flow rate of fluid in real time, namely, the peristaltic pump body can be programmed to control the fluid conveying at constant speed and constant flow. The communication interface of the peristaltic pump can also be used as an interface for communicating with a superior unit on a production line, and the superior control system is used for expanding the communication interface and controlling the flow and the flow rate of the fluid. One of the functions of the peristaltic pump is to be able to deliver fluid (suspension) to the turbulence promoter 62 connected in series between the peristaltic pump and the light energy receiving glass tube 2 at a constant speed and flow rate; and the second function is to communicate with the superior control system, and the peristaltic pump body does not contact with the fluid and will not pollute the fluid (suspension). The flow speed and the flow can be controlled after the fluid enters the turbulence generator and the generated flow state is changed, so that the suspension entering the light energy receiving glass tube 2 realizes that micron-sized solid spore particles can be vertically turned in the narrow flow field of the glass groove tube. The dense spore group is irradiated by laser energy at the fastest displacement rate to break the wall, so that the spore can be broken by the laser energy, and the spore inclusion is not coked due to overlong irradiation time.

The turbulence promoters 62 are connected in series between the peristaltic pump and the light energy receiving glass tube 2, the turbulence promoters 62 being arranged primarily to make the fluid entering the light energy receiving glass tube 2 generate turbulence. The turbulence promoter 62 is fabricated from a custom-made glass with single component of silica dioxide into the shape of a thin flat glass channel, and protrusions (first and second partitions 71, 72) with a certain interval of glass are welded in the glass channel. When the fluid flows through the first and second partitions 71, 72, the flow direction of the fluid is constantly changed, thereby promoting the formation of turbulence. The turbulence promoters 62 of glass material do not contaminate the material.

Referring to FIG. 1 to 7, the isolation box type foam isolator 3 comprises a second water tank 32 and a foam isolation chamber 31, and a through hole 10 is formed between the second water tank 32 and the foam isolation chamber 31. One end of the light energy receiving glass tube 2 is connected to the first water tank 621, the other end of the light energy receiving glass tube is connected to the second water tank 32, and the second water tank 32 is communicated with the foam isolation chamber 31 through the through hole 10. A foam outlet 5 and a finished product stock solution outlet 4 are arranged in the foam isolation chamber 31, and the foam outlet 5 is positioned at the other side of the foam isolation chamber 31 relative to the through hole 10.

In the above technical solution, the light receiving glass tube 2 is in the shape of a thin and flat hollow, and is made of glass with single component of silicon dioxide and high-transmittance, and is divided into two radiation-receiving surfaces and two side surfaces. The spacing distance between the irradiation surfaces of the light energy receiving glass tube 2 is determined by the type of the matched laser 14 and the light emitting parameters set by the laser, the types of the material spores, and the speed and the flow rate of the material fluid. Similarly, the size of the irradiated surface of the light energy receiving glass tube 2 must meet the technical parameter requirement of receiving the irradiation from the laser 14. Since the light beam of the laser 14 is different monochromatic light, the light transmittance is substantially completely determined by the material of the glass. The glass material of the light energy receiving glass tube 2 is required to be a glass material which has good light transmittance in the whole spectrum band, low refractivity, small thermal expansion coefficient, high chemical thermal stability, smoothness and easy disinfection and cleaning. When laser irradiates glass, part of the laser penetrates through the glass, and part of the laser is reflected out of the surface of the glass. Therefore in order to improve the spore wall breaking efficiency, transparent glass with the light transmittance higher than 95% on average is required to be selected. Since the light energy receiving glass tube 2 is symmetrically designed and manufactured, two light energy receiving surfaces are divided into A, B surfaces. If the A surface is irradiated by laser for a long time and the glass frosted in the irradiated part of the glass (roughened by Rayleigh effect of laser), the B surface can be simply replaced as the laser irradiation receiving surface.

The size of the spores is different, for example, the average size of ganoderma spores is 6×9 um; the average size of pine pollen spores was 35×50 um. The mass percentage of the spores in the suspension is determined according to the types and sizes of the spores subjected to wall breaking treatment when the suspension material is prepared. The distance between the irradiation receiving surfaces of the light energy receiving glass tube 2 can be customized in advance according to the types of spores in the suspension. Under the effect of the turbulence generator, the suspension fluid entering the light energy receiving glass tube 2 presents a turbulent flow state, namely a turbulent flow state, and solid particle spores in the suspension roll in a narrow space of the light energy receiving glass tube 2, so that all the particles of spores can be ieeadiated to break the walls as much as possible.

Function of the isolation box type foam isolator 3: the micro bubbles always exist in the suspension fluid, insoluble micro bubbles in the suspension fluid are difficult to escape outwards due to the influence of a relatively stable liquid film on the surface of the suspension. Bubble aggregates generated by heat can form foam when materials are irradiated by laser, and the stability of the foam is related to the viscosity of the fluid, the elastic action of the surface and the rheological property of the surface. After the fluid is irradiated by the laser light energy through the interlayer of the light energy receiving glass tube 2, the micro bubbles in the fluid are gathered into foam by the thermal effect and are accumulated on the upper layer of the fluid, and the excessive foam seriously influences the efficiency of laser wall breaking, therefore the invention provides the isolation box type foam isolator 3 made of the glass with single component of silicon dioxide.

If the fluid flowing out from the first light energy receiver has a large amount of foams caused by spore wall breaking and temperature rising, it will have a bad influence on the wall breaking efficiency of laser irradiation of spores of the light energy receivers connected in series after the station, and the foam isolator can better solve the problem. By observing the foam with a microscope, a plurality of fine spore exine shell crumbles still exist in the foam, and Separating the broken spore exine shell crumbles from the fluid through the foam isolatoris of great help to improve the spore wall breaking efficiency of the next light energy receiver.

When the fluid system flows, the fluid system with the boundary layer is divided into three flow states of laminar flow, transitional flow and turbulent flow. When the fluid flows to the input end of the light energy receiver through the conveying pipeline system of production line, after the fluid is conveyed by the pipeline system, materials in the pipeline gradually appears the laminar flow state under the influence of gravity and other factors, and most spores are deposited at the lower part of the fluid in the pipeline. At the moment, in order to improve the efficiency of laser wall breaking method and avoid the damage to spore inclusions during the laser wall breaking, the fluid state is very necessary to be changed into the turbulent flow state. The invention firstly conveys the prepared materials, namely suspension of spores and pure water, to the input end of the light energy receiver by a pipeline system, and the materials are extracted by a station control system at a preset speed and a preset flow (or at a preset speed and a preset flow by a peristaltic pump) and then are conveyed to the input end of the light energy receiving glass tube 2 by the turbulence promoter 62, so that the flow speed and the flow of the fluid entering the light energy receiving glass tube 2 are accurately controlled. The turbulence promoters 62 cause the fluid entering the light energy receiving glass tube 2 in a turbulent state.

Referring to FIG. 1 to 7, in order to achieve a good wall breaking effect, preferably, a plurality of light energy receivers are provided, and are sequentially connected in series along the conveying direction of the suspension, and each light energy receiver is provided with a laser 14 and an XY limiter 16 corresponding to the light energy receiver. The XY limiter 16 is an electric or manual XY translation stage, and since the XY stopper 16 is in the prior art, the detailed structure and the working principle of the XY limiter 16 are not described herein. When the light energy receivers are connected in series, the finished product stock solution outlet 4 of the upper light energy receiver is in pipeline connection with the feed port 8 of the turbulence promoting chamber 622 of the lower light energy receiver, the foam sediment pipeline 15 is arranged in the light energy receiving area, and the foam outlet 5 of each light energy receiver is independently connected with the foam sediment pipeline 15. According to the specific wall breaking condition and requirement, one optical energy receiving area can be connected with any number of optical energy receivers in series. The laser 14 is used for horizontally irradiating the light energy receiving glass tube 2, namely the light energy receiving glass tube 2 is vertically installed and the laser 14 is installed at one side of the light energy receiving glass tube 2 in the horizontal direction, so that the laser 14 horizontally emits light.

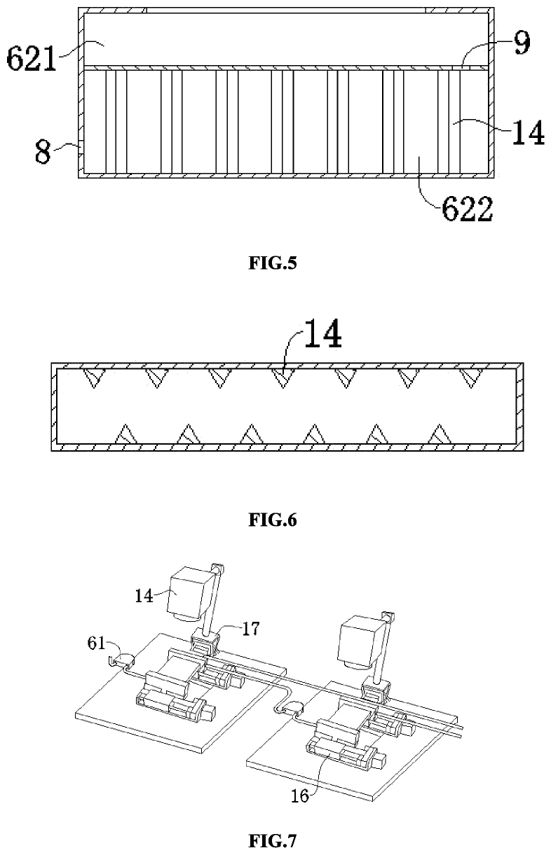

Example 2, a production line assembly and production line for laser wall breaking of plant spores, referring to FIG. 5 to 6, differs from example 1 in that the first partition plate 71 and the second partition plate 72 in the turbulence promoting chamber 622 are replaced by raised stripes. The method comprises the following specific steps: the turbulence promoting chamber 622 is provided with raised stripes inside, the crisscross setting of raised stripes is in the opposite sides of the inner wall of the turbulence promoting chamber 622, the cross section of the raised stripes is triangle-shaped and arranged between the feed port 8 and the perforations 9 of the turbulence promoting chamber 622.

Example 3, a production line assembly for laser wall breaking of plant spores, referring to FIG. 7, differs from example 1 in that the laser 14 is used to irradiate a light energy receiving glass tube 2 from top to bottom, i.e. the light energy receiving glass tube 2 is horizontally installed and the laser 14 is installed at one side above the light energy receiving glass tube 2, so that the laser 14 looks down to emit light. The bottom of the laser 14 is provided with a Z limiter 17, and the Z limiter 17 is a Z-direction electric/manual lifting table. The Z limiter 17 is used for driving the laser 14 to move up and down so as to position the specified position of the light spot in the irradiation surface of the thin flat type light energy receiving glass tube 2.

Example 4 also provides a production line for laser wall breaking of the plant spores, which comprises a raw material inspection and disinfection module 20, a material preparation module 21, a production line assembly and an inspection and subpackage module 23.

It should be noted that the production line assembly in this example adopts a specific technical solution for the production line assembly for laser wall breaking of plant spores in the foregoing example 1 to 3.

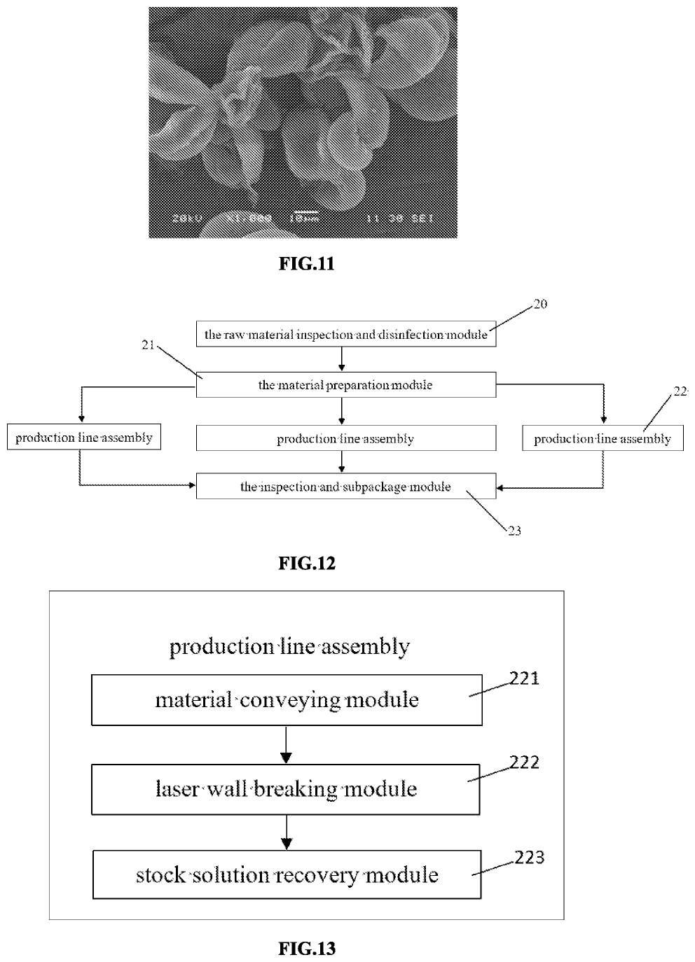

Referring to FIG. 11-12, the raw material inspection and disinfection module 20 is used to inspect, sterilize, and store plant spore material. The raw material inspection and disinfection module 20 comprises a raw material inspection room, a disinfection room and a storage warehouse, and the plant spore raw material is inspected by the raw material inspection room, conveyed to the disinfection room for sterilization and disinfection, and finally conveyed to the storage warehouse for storage. As the inspection of physicochemical indexes of pesticide residues and the inspection of excessive heavy metals are finished in a production place, the inspection of the spore raw materials in storage mainly aims at the inspection of microbial pollution. For example, the indexes of mould in bacteria attached to Ganoderma lucidum spore raw materials are most easily out of standard, and spores which are not sterilized are easily changed in quality during storage. The raw material inspection room is responsible for detecting main biochemical pollution indexes in the raw materials and issuing an inspection report. The solution is adopted by the disinfection room according to the results reported by the inspection, for example, by using ultraviolet lamp sterilization treatment method, the content of various microorganisms can be controlled by controlling the irradiation time and the irradiation power of the ultraviolet lamp and turning over spores appropriately, so that the stored plant spores are not deteriorated in the storage period.

Referring to FIG. 11 to 12, the material preparation module 21 is used for mixing plant spores and pure water to prepare a solid-liquid suspension material, that is, the suspension to be subjected to laser wall breaking. The material preparation module 21 is a material preparation barrel, and the function of the material preparation barrel is to depolymerize the agglomeration of ultrafine particles such as plant spores and fully stir the suspension of the spores and water in a turbulent flow and a pulsating manner, so that the insoluble ultrafine solid particle spores are fully dispersed into liquid to form the suspension, and dissolved gas attached to the surface of the spore pits is discharged as much as possible. Therefore, the material preparation barrel is a barrel body with a stirring function, and the material preparation link is one of the main links that a biological factory for breaking the wall of the plant spores can maintain stable spore wall breaking rate.

Referring to FIG. 11-12, the inspection and subpackage module 23 is used for inspecting and subpackaging the wall-broken finished stock solution. The inspection and subpackage module 23 comprises a finished product inspection room, a subpackaging room and a finished product warehouse, and after the finished product stock solution subjected to wall breaking is inspected to be qualified by the finished product inspection room, the finished product stock solution is respectively subpackaged in the subpackaging room and finally conveyed to the finished product warehouse for storage. The finished product inspection room is a checkpoint for products leaving the factory, and inspectors regularly take materials from the material from the pipe intake of this module for biochemical inspection and print batch numbers. The subpackaging room room is provided with a small-sized oxygen-insulated vacuum subpackaging room and a subpackaging barrel isolation room which are separated by visual glass, and an ultraviolet lamp is required to irradiate the sterilizing device and a nitrogen charging port, and a gas output port of the nitrogen making machine is connected with the nitrogen charging port. The subpackaging barrel (25 Kg/barrel or other standard capacity barrel) is filled with nitrogen in advance and pushed into the isolation chamber of the subpackaging barrel one by one. And performing operation and subpackage by using silica gel gloves reserved in an oxygen-insulated vacuum subpackaging room with small visual glass intervals by subpackage operators (the operators stretch the silica gel gloves into the subpackaging room to perform subpackage operation by using handles outside the subpackaging room). The finished product barrel pipeline of the module is provided with a constant volume electromagnetic valve capable of setting split charging capacity, and the stock solution is automatically filled according to the set filling capacity. The filled subpackaging barrel is taken out through the subpackaging isolation room and is conveyed to a storage storehouse, and oxidation and secondary pollution caused by air entering the subpackaging barrel are avoided.

The present invention is not limited to the above-mentioned alternative embodiments, and any other various products can be obtained by anyone in the light of the present invention, but no matter what changes are made in its shape or structure, all technical solutions that fall within the scope defined by the claims of the invention are within the protection scope of the invention.

1. A production line assembly for laser wall breaking of plant spores, wherein the production line assembly comprises a material conveying module, a laser wall breaking module and a stock solution recovery module which are successively connected through pipelines; the material conveying module is configured to convey suspension to be subjected to laser wall breaking to the laser wall breaking module;
the laser wall breaking module is formed by sequentially connecting a plurality of light energy receiving areas along the conveying direction of the suspension to be subjected to laser wall breaking;
the light energy receiving area comprises a laser, a light energy receiver and an XY limiter; the suspension liquid to be subjected to laser wall breaking flows in the light energy receiver, and the laser correspond to the light energy receiver one by one and is configured to perform laser wall breaking on the suspension liquid to be subjected to laser wall breaking; the laser is configured to horizontally irradiate the light energy receiver, or the laser is configured to irradiate the light energy receiver from top to bottom;
the light energy receiver is mounted on the XY limiter, and the XY limiter is configured to adjust the position of the light energy receiver in X-axis and Y-axis direction;
the light energy receiver comprises a turbulence generator, a light energy receiving glass tube and an isolation box type foam isolator which are sequentially connected along the conveying direction of the suspension to be subjected to laser wall breaking;
the turbulence generator comprises turbulence promoter and peristaltic pump; the turbulence promoter, the light energy receiving glass tube and the isolation box type foam isolator are sequentially communicated and integrally arranged, one side of the peristaltic pump is connected with turbulence promoter through a pipeline, the other side of peristaltic pump is connected with conveying pipe, which is configured to convey the suspension to be subjected to laser wall breaking into the turbulence promoter;
the turbulence promoter comprises a box body, a turbulence promoting chamber and a first water tank are arranged in the box body, a through hole is arranged between the first water tank and the turbulence promoting chamber, the first water tank is connected with one end of the light energy receiving glass tube and is communicated with the turbulence promoting chamber through the through hole, the turbulence promoting chamber is provided with a feed port relative to the other end of the through hole, and the pump is connected with the feed port.

2. The production line assembly for laser wall breaking of plant spores of claim 1, wherein: the material conveying module comprises a material preparation barrel and a conveying pipe, and the conveying pipe is configured to extract the suspension to be subjected to laser wall breaking in the material preparation barrel and convey the suspension to the light energy receiver.

3. (canceled)

4. (canceled)

5. The production line assembly for laser wall breaking of plant spores of claim 4, wherein: partition plates are arranged in the turbulence promoting chamber, the partition plates comprise the first partition plate and the second partition plate which are respectively fixed on two opposite sides of the inner wall of the turbulence promoting chamber, the first partition plate and the second partition plate are respectively provided in plurality and are arranged between the feed port of the turbulence promoting chamber and the perforation.

6. The production line assembly for laser wall breaking of plant spores of claim 4, wherein: the turbulence promoting chamber is provided with raised stripes inside and the crisscross setting of raised stripes is in the two opposite sides of the inner wall of the turbulence promoting chamber, the cross section of the raised stripes is triangular-shaped and arranged between the feed port and the perforation of the turbulence promoting chamber.

7. The production line assembly for laser wall breaking of plant spores of claim 4, wherein: the isolation box type foam isolator comprises a second water tank and a foam isolation chamber, a through hole is formed between the second water tank and the foam isolation chamber, one end of the light energy receiving glass tube is connected with the first water tank, the other end of the light energy receiving glass tube is connected with the second water tank, the second water tank is communicated with the foam isolation chamber through the through hole, and a foam outlet and a finished product stock solution outlet are arranged in the foam isolation chamber.

8. A production line for laser wall breaking of the plant spores, wherein the production line comprises a raw material inspection and disinfection module, a material preparation module, a production line assembly for laser wall-breaking of plant spores according to claim 1 and an inspection and subpackage module.

9. The production line for the laser wall breaking of plant spores of claim 8, wherein: the raw material inspection and disinfection module is configured to inspect, disinfect, and store plant spore raw materials, the material preparation module is configured to mix plant spores and pure water, the suspension to be subjected to laser wall breaking is prepared, and the inspection and subpackage module is configured to inspect and subpackage to the finished product stock solution after wall breaking.

10. The production line for the laser wall breaking of plant spores of claim 9, wherein: the raw material inspection and disinfection module comprises a raw material inspection room, a disinfection room and a storage warehouse, and the plant spore raw material is inspected by the raw material inspection room, conveyed to the disinfection room for sterilization and disinfection, and finally conveyed to the storage warehouse for storage;
the inspection and subpackage module comprises a finished product inspection room, a subpackaging room and a finished product storehouse, and after the finished product stock solution subjected to wall breaking is inspected to be qualified by the finished product inspection room, the finished product stock solution is respectively subpackaged in the subpackaging room and finally conveyed to the finished product storehouse for storage.
